(19) United States
(12) Patent Application Publication
(10) Pub. No.: US 2021/0000361 A1
(43) Pub. Date: Jan. 7, 2021

(54) APPARATUS AND METHOD OF MONITORING INTRA-ABDOMINAL PRESSURE AND URINE OUTPUT

(71) Applicant: SERENNO MEDICAL, Yokneam Ilit (IL)

(72) Inventors: Tomer LARK, Herzeliya (IL); Noam HADAS, Tel Aviv (IL)

(21) Appl. No.: 16/767,712

(22) PCT Filed: Nov. 29, 2018
(86) PCT No.: PCT/IL2018/051312

§ 371 Date: May 28, 2020

Related U.S. Application Data
(60) Provisional application No. 62/591,797, filed on Nov. 29, 2017.

(51) Int. Cl. A61B 5/03; A61B 5/20; A61B 5/00

(52) U.S. Cl. CPC A61B 5/036; A61B 5/208; A61B 2562/0247; A61B 5/6852; A61B 5/205

(57) ABSTRACT

A device for measuring a rate of production of urine in a patient comprising a catheter; a pressure transducer; and a means for measuring the amount of urine produced. Urine flow from the bladder is prevented until a predetermined pressure is reached in the bladder. Urine flow is then allowed; the bladder pressure and the volume of urine exiting the bladder are measured. When the bladder pressure reaches a second, lower predetermined pressure, urine flow is again prevented. From the measured pressure during urine flow and from the volume of urine exiting the bladder, the intra-abdominal pressure and the urine production rate can be determined.

### FIELD OF THE INVENTION

The invention relates generally to medical monitoring and measuring devices, and more specifically to physiological parameter measurement equipment. The invention relates particularly to apparatus configured as an assembly to measure a hospitalized patient's urine output and intra-abdominal pressure.

### BACKGROUND OF THE INVENTION

It is well established in the medical literature that urine output (UO) of a patient has diagnostic usefulness as a tool to assess progress of the patient's recovery from a medical condition. For example, a gradual decrease in a patient's UO over time will usually indicate increased risk for kidney failure. By monitoring the patient's UO, the physician or medical support staff may gain advance warning of complications which may affect the patients' health and safety. In such cases, adequate counter-measures can be employed well before other physiological parameters of the patient demonstrate signs of distress, and before life-threatening conditions develop.

Urine output is usually measured by a graduated urine collection bag, with the fluid level in the bag read periodically by a nurse. The quantity of urine is recorded, and the difference from the last reading divided by time since the last reading serves as the rate of urine production. When the urine collection bag is full it is replaced with a new one. Since the bag is flexible and doesn't have a fixed shape, volume readings are not accurate. If more accurate readings are needed, special urine measuring bags are used. These bags include a hard plastic cuvette with a well-defined volume and clear graduations. The urine flows from the tube into a cuvette where the liquid level can be read to higher precision. After each reading, the cuvette is tilted to empty it into the bag. This method allows for more accurate readings, but the special bags are more expensive and still require manual labor. A method of producing such bags is described by Lawrence Salvadori, Dennis Schreuer in U.S. Pat. No. 7,645,968, but many other techniques and designs are available.

Many techniques have been proposed to automate and increase the accuracy of UO measurement, which are generally divided into three categories. One is a method where the urine flows into a cuvette of known and fixed cross-section, where its level is monitored by some form of electronic sensor, such as capacitive sensors as in U.S. Pat. No. 6,125,696, an optical sensor as in US20120078137, or an ultrasonic level meter. In all these systems, when the measuring volume is full, a valve is automatically opened to allow the volume to empty into the urine collection bag.

Another method uses a drop counter, as in US 20100274217, where the urine is made to flow through a small orifice producing drops of fixed volume, with a sensor to count each drop.

A third method measures urine output by weighing the urine collection bag as in US 20100280443. The bag may be resting on the scale or may be hanging from it.

It is therefore a long felt need to provide a device for measuring IAP and trends in IAP which does not place a patient at risk of infection (specifically UTI) or require tiresome manual adjustment of a plurality of tubing devices.

### SUMMARY OF THE INVENTION

It is an object of the present invention to disclose a system and method of measuring IAP and trends in IAP monitoring intra-abdominal pressure (IAP) and urine output (OU), typically in a human patient, which relies on controlled transfer of urine from the bladder to the collection bag rather than depending on free flow of the urine from the bladder.

It is another object of the present invention to disclose a device for measuring a rate of production of urine in a patient comprising:

a catheter;

a pressure sensor, either in direct fluid connection with the urine in the input port of the pump, or through a very thin, isolating film membrane;

a urine amount sensor, said urine amount sensor selected from a group consisting of a flow sensor, a volumetric pump and any combination thereof; and

a processor configured to control:
occlusion of a said catheter until a predetermined first pressure within said lumen is detected;
emptying of said bladder into a urine bag by means of at least one member of a group consisting of: passively flowing urine from said bladder and actively pumping urine from said bladder;
measuring an amount of urine flowing through said lumen until a predetermined second pressure within said lumen is detected;
calculating an instantaneous urine production rate by dividing said measured amount of urine by a time period, said time period being either a time from a first occlusion of said lumen or a time from a last emptying of said bladder;
repeating steps (a) to (e) at least once; and
generating an average urine production rate by averaging at least two values of said instantaneous urine production rate.

It is another object of the present invention to disclose the device, wherein said predetermined first pressure is in a range from 3 mmHg to 70 mmHg.

It is another object of the present invention to disclose the device, wherein said predetermined second pressure is in a range from 0 mmHg to 5 mmHg.

It is another object of the present invention to disclose a device for measuring a rate of production of urine in a patient comprising:

a catheter;

a pressure sensor;

a urine amount sensor, said urine amount sensor selected from a group consisting of a flow sensor, a volumetric pump and any combination thereof; and

a processor configured to control:
occluding a lumen of said catheter for a predetermined time period;
emptying said bladder into a urine bag by means of at least one member of a group consisting of: passively flowing urine from said bladder and actively pumping urine from said bladder;
measuring an amount of urine flowing through said lumen until a predetermined pressure within said lumen is detected;
calculating an instantaneous urine production rate by dividing said measured amount of urine by a time period, said time period being either a time from a first occlusion of said lumen or a time from a last emptying of said bladder;
repeating steps (a) to (e) at least once; and
generating an average urine production rate by averaging at least two values of said instantaneous urine production rate.

It is another object of the present invention to disclose the device, wherein said predetermined pressure is in a range from 0 mmHg to 5 mmHg.

It is another object of the present invention to disclose a device for measuring a rate of production of urine in a patient comprising:

a catheter;

a pressure sensor;

a urine amount sensor, said urine amount sensor selected from a group consisting of a flow sensor, a volumetric pump and any combination thereof; and

a processor configured to control:
occluding a lumen of said catheter;
emptying said bladder into a urine bag by means of at least one member of a group consisting of: passively flowing urine from said bladder and actively pumping urine from said bladder until a predetermined pressure within said lumen is detected;
during said emptying: (1) measuring an amount of urine removed from said bladder; (2) measuring pressure within said lumen; and (3) storing said measured amount of urine removed and said measured pressure; and
said measured amount of urine removed being greater than a predetermined amount, said IAP is calculated as said measured pressure.

It is another object of the present invention to disclose the device, wherein said predetermined pressure is in a range from 0 mmHg to 5 mmHg.

It is another object of the present invention to disclose the device, wherein said step of emptying said bladder comprises a plurality of steps, each of said plurality of steps removing from said bladder an amount of urine in a range between 0.1 cc and 5 cc.

It is another object of the present invention to disclose the device, additionally comprising a step of determining an adjusted instantaneous IAP from a lowest measured pressure, thereby removing an effect on said urine pressure of respiratory-induced pressure oscillations.

It is another object of the present invention to disclose a device for measuring a rate of production of urine in a patient comprising:

a catheter;

a pressure sensor;

a urine amount sensor, said urine amount sensor selected from a group consisting of a flow sensor, a volumetric pump and any combination thereof;

a processor configured to control:
occluding a lumen of said catheter until a predetermined pressure within said lumen is detected;
emptying said bladder into a urine bag by means of at least one member of a group consisting of: passively flowing urine from said bladder and actively pumping urine from said bladder;
measuring an amount of urine flowing through said lumen; and
during said emptying: (1) measuring an amount of urine removed from said bladder; (2) measuring pressure within said lumen; and (3) storing said measured amount of urine removed and said measured pressure; and

said measured amount of urine removed being greater than a predetermined amount, said IAP is calculated as said measured pressure.

It is another object of the present invention to disclose the device, wherein said predetermined pressure is in a range from 1 mmHg to 15 mmHg.

It is another object of the present invention to disclose the device, wherein said step of emptying said bladder comprises a plurality of steps, each of said plurality of steps removing from said bladder an amount of urine in a range between 0.1 cc and 5 cc.

It is another object of the present invention to disclose the device, additionally comprising a step of determining an adjusted instantaneous IAP from a lowest measured pressure, thereby removing an effect on said urine pressure of respiratory-induced pressure oscillations.

It is another object of the present invention to disclose a device for measuring a rate of production of urine in a patient comprising:

a catheter;

a pressure sensor;

a urine amount sensor, said urine amount sensor selected from a group consisting of a flow sensor, a volumetric pump and any combination thereof; and

a processor configured to control:
occluding passage of urine from a bladder of said patient;
real-time measuring pressure in said bladder while said passage of urine is occluded;
storing said measured pressure as a function of time;
detecting at least one time period in which a minimum pressure at an end of an expiratory phase of said patient's breathing is in a range between 5mmHg and 40 mmHg;
determining a dwell pressure, said dwell pressure being a minimum pressure in a central portion of said at least one time period;
occluding said passage of urine until said minimum pressure at an end of an expiratory phase of said patient's breathing is in a range between 30% and 50% above said dwell temperature;
releasing urine from said bladder; during said release: (1) real-time measuring an amount of urine released; real-time measuring said pressure in said bladder; comparing said pressure in said bladder to a predetermined pressure, said predetermined pressure being in a range between 0% and 30% below said dwell pressure;
said pressure being below said predetermined pressure, (1) occluding said passage of urine; and determining said IAP to be said dwell pressure; and
determining said UO to be said amount of urine released.

It is another object of the present invention to disclose a method for measuring a rate of production of urine in a patient comprising steps of:

inserting a catheter into said patient's bladder;

occluding a lumen of said catheter until a predetermined first pressure within said lumen is detected;

emptying said bladder into a urine bag by means of at least one member of a group consisting of: passively flowing urine from said bladder and actively pumping urine from said bladder;

measuring an amount of urine flowing through said lumen until a predetermined second pressure within said lumen is detected;

calculating an instantaneous urine production rate by dividing said measured amount of urine by a time period, said time period being either a time from a first occlusion of said lumen or a time from a last emptying of said bladder;

repeating steps (a) to (e) at least once; and generating an average urine production rate by averaging at least two values of said instantaneous urine production rate.

It is another object of the present invention to disclose the method, wherein said predetermined first pressure is in a range from 3 mmHg to 70 mmHg.

It is another object of the present invention to disclose the method, wherein said predetermined second pressure is in a range from 0 mmHg to 5 mmHg.

It is another object of the present invention to disclose the method, wherein said step of measuring said amount of urine is performed by means of a member of a group consisting of a flow sensor, a volumetric pump and any combination thereof.

It is another object of the present invention to disclose a method for measuring a rate of production of urine in a patient comprising steps of:

inserting a catheter into said patient's bladder;

occluding a lumen of said catheter for a predetermined time period;

emptying said bladder into a urine bag by means of at least one member of a group consisting of: passively flowing urine from said bladder and actively pumping urine from said bladder;

measuring an amount of urine flowing through said lumen until a predetermined pressure within said lumen is detected;

calculating an instantaneous urine production rate by dividing said measured amount of urine by a time period, said time period being either a time from a first occlusion of said lumen or a time from a last emptying of said bladder; repeating steps (a) to (e) at least once; and

generating an average urine production rate by averaging at least two values of said instantaneous urine production rate.

It is another object of the present invention to disclose the method, wherein said predetermined pressure is in a range from 0 mmHg to 5 mmHg.

It is another object of the present invention to disclose the method, wherein said step of measuring said amount of urine is performed by means of a member of a group consisting of a flow sensor, a volumetric pump and any combination thereof.

It is another object of the present invention to disclose a method for measuring intra-abdominal pressure (IAP) in a patient comprising steps of:

inserting a catheter into said patient's bladder;

occluding a lumen of said catheter;

emptying said bladder into a urine bag by means of at least one member of a group consisting of: passively flowing urine from said bladder and actively pumping urine from said bladder until a predetermined pressure within said lumen is detected;

during said emptying:
measuring an amount of urine removed from said bladder;
measuring pressure within said lumen; and
storing said measured amount of urine removed and said measured pressure; and

said measured amount of urine removed being greater than a predetermined amount, said IAP is calculated as said measured pressure.

It is another object of the present invention to disclose the method, wherein said predetermined pressure is in a range from 0 mmHg to 5 mmHg.

It is another object of the present invention to disclose the method, wherein said step of emptying said bladder comprises a plurality of steps, each of said plurality of steps removing from said bladder an amount of urine in a range between 0.1 cc and 5 cc.

It is another object of the present invention to disclose the method, additionally comprising a step of determining an adjusted instantaneous IAP from a lowest measured pressure, thereby removing an effect on said urine pressure of respiratory-induced pressure oscillations.

It is another object of the present invention to disclose a method of measuring intra-abdominal pressure (IAP) in a patient comprising steps of:

inserting a catheter into said patient's bladder;

occluding a lumen of said catheter until a predetermined pressure within said lumen is detected;

emptying said bladder into a urine bag by means of at least one member of a group consisting of: passively flowing urine from said bladder and actively pumping urine from said bladder;

measuring an amount of urine flowing through said lumen;

during said emptying:
measuring an amount of urine removed from said bladder;
measuring pressure within said lumen; and
storing said measured amount of urine removed and said measured pressure; and

said measured amount of urine removed being greater than a predetermined amount, said IAP is calculated as said measured pressure.

It is another object of the present invention to disclose the method, wherein said predetermined pressure is in a range from 5 mmHg to 40 mmHg.

It is another object of the present invention to disclose the method, wherein said step of emptying said bladder comprises a plurality of steps, each of said plurality of steps removing from said bladder an amount of urine in a range between 0.1 cc and 5cc.

It is another object of the present invention to disclose the method, additionally comprising a step of determining an adjusted instantaneous IAP from a lowest measured pressure, thereby removing an effect on said urine pressure of respiratory-induced pressure oscillations.

It is another object of the present invention to disclose a method of measuring urine output (UO) intra-abdominal pressure (IAP) in a patient comprising steps of:

occluding passage of urine from a bladder of said patient;

real-time measuring pressure in said bladder while said passage of urine is occluded;

storing said measured pressure as a function of time;

detecting at least one time period in which a minimum pressure at an end of an expiratory phase of said patient's breathing is in a range between 5 mmHg and 40 mmHg;

determining a dwell pressure, said dwell pressure being a minimum pressure at a center of said at least one time period;

occluding said passage of urine until said minimum pressure at an end of an expiratory phase of said patient's breathing is in a range between 30% and 50% above said dwell temperature;

releasing urine from said bladder; during said release:

real-time measuring an amount of urine released;

real-time measuring said pressure in said bladder; and

comparing said pressure in said bladder to a predetermined pressure, said predetermined pressure being in a range between 0% and 30% below said dwell pressure; and

said pressure being below said predetermined pressure, performing the following steps:

occluding said passage of urine;
determining said IAP to be said dwell pressure; and
determining said UO to be said amount of urine released.

### BRIEF DESCRIPTION OF THE FIGURES

In order to better understand the invention and its implementation in practice, a plurality of embodiments will now be described, by way of non-limiting example only, with reference to the accompanying drawings, wherein

FIG. 1 schematically illustrates pressure in a bladder as a function of time;

FIG. 2 illustrates a block diagram of a preferred embodiment of a hardware configuration needed to implement the method in a UO/IAP medical measuring device;

FIG. 3 schematically illustrates a typical pressure/volume vs. time graph in a UO continuous measuring mode when using a peristaltic pump;

FIG. 4 illustrates a typical pressure/volume vs. time graph in a UO continuous measuring mode, when using a fixed stroke volume pump;

FIG. 5 illustrates a typical pressure/time graph in a mode preserving a natural urination cycle;

FIG. 6-7 schematically illustrate a pressure/time graph in an embodiment of an IAP measuring cycle; and

FIG. 8 illustrates an embodiment of a method for measuring both UO and IAP in a real-time, continuous mode.

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for monitoring intra-abdominal pressure (IAP) and urine output (OU), typically in a human patient, that relies on controlled transfer of urine from the bladder to the collection bag rather than depending on free flow of the urine from the bladder.

### Drawbacks of the Prior Art Devices, Systems and Methods

Prior art methods have drawbacks, such as:

a. Free flowing urine may form disconnected sections of urine-filled tube, if the tube does not slope down along its entire length. In such a case, airlocks form which prevent the urine from flowing to the measuring device until the entire loop is filled, disrupting the continuous measuring process.

b. They are affected by urine composition, which may be very different from patient to patient. Urine consistency and color may vary and thus may cause errors in measurement of optical or drop-based sensors.

c. All methods that collect urine in a container which is periodically emptied lose accuracy as residues accumulate in the container, decreasing its effective volume.

d. Drop counting and weighing methods lose accuracy if the tube or device is moved during measurement, forcing smaller drops to drop, or adding noise to the weigh reading.

e. Level measuring devices lose accuracy if not installed perfectly vertical.

f. The urine collection bag must be placed below the level of the patient, or urine will not flow to the bag. They all relay on gravity and consistent downward sloping tubing from the catheter to the measuring device to move the urine in the tube.

Techniques employed to non-invasively monitor a patient's intra-abdominal pressure are configured to measure the pressure of fluid contained within the patient's bladder and are normally only performed once every several hours. When properly performed, such techniques can indicate to a high level of accuracy the pressure in the patient's abdominal cavity.

The current standard for measuring a patient's intra-abdominal pressure involves disassembling a urinary catheter drain tube from a urine catheter located inside the patient's bladder, and injecting a known volume of sterile saline through the catheter and into the patient's bladder. (For convenience, all types of urinary catheter will generally be referred to as a “catheter”). The catheter port is then connected to a pressure sensor, which provides the intra-abdominal pressure at that instant. The catheter is then re-connected to the drain and the bladder emptied.

Various methods for performing these actions have been developed over the years, involving either three-lumen catheters, passing a needle though the catheter's aspiration port for injecting the saline bolus and connecting the pressure sensors, or using one of multiple variations of tubing systems comprising clamps, stopcocks, needles and valves, and/or some other plumbing accessories.

Unfortunately, any opening in the closed urine drainage system, and certainly injecting fluids into the patient's bladder, place both the patient and the health care practitioner at increased risk of infection. The patient is at risk of infection both by microbes introduced when the circuit is breached as well as due to bacteria present on the internal surfaces of the tubes being washed into the bladder by the reverse flow.

If performed manually, the process is labor intensive and slow. Even if performed by an automatic device, it still requires the replenishment of the external reservoir.

Such methods only measure intra-abdominal pressure (IAP) at the moment of sampling (filling, measuring and emptying the bladder) and cannot maintain continuous measurement or redo the process in a short time frame. There is also the issue of the cost of the tubing arrangement, which increases with the complexity of the system.

Unlike U.S. Pat. No. 5,385,563, WO2004080519A1, EP0258690A2, US20020065472, this invention does not require use of a special catheter, and any standard type of urinary catheters is suitable.

Unlike U.S. Pat. Nos. 5,865,764 A, 6,503,208, the present invention does not need an external fluid (saline) source to fill the bladder in order to measure at the correct volume.

It is therefore a long felt need to provide a device for measuring IAP and trends in IAP which does not place a patient at risk of infection (specifically UTI) or require tiresome manual adjustment of a plurality of tubing devices, such as two-way valves or stopcocks.

The device comprises a no flow-through controlled metering pump connected between a urinary catheter and a urine collection bag and a pressure transducer fluidly coupled with the urine in the catheter, or any other mean of measuring the pressure at the pump inlet port connected to the urine catheter.

Preferably, the pump and the pressure transducer are enclosed in a housing configured to be applied on a patient's leg close to or abutting the catheter port, with minimal discomfort. In some embodiments, the pump is located near the patient and pressure is measured by a remote pressure transducer separate from the pump and in fluid communication with the pump inlet port via a long tube, or by employing a pump that allows inlet pressure determination using other means.

A processor receives measurements in real time from the pressure transducer, the measurements representing the pressure inside the bladder, and activates the pump as needed to move urine from the bladder to the collection bag, in order to measure urine output and intraabdominal pressure. In preferred embodiments, the control device is not mounted on the patient's body. The control device can be in wired or wireless communication with the pump and pressure transducer.

In order to measure UO, a controlled pump is connected between the urine catheter and the urine collection tube leading to the urine collection bag. Additionally, a fluid pressure sensor is fluidly coupled to the inlet port of the pump, hence it is in fluid connection with the volume of the bladder through the lumen of the catheter. The pump is a positive displacement pump such as a membrane pump or a piston pump, having active valves so that at there is never a possibility of free flow through the pump from the catheter to the bag.

To measure either UO, IAP, or both, the processor determines pressure from the pressure sensor readings and actuates the pump, as needed. Several embodiments of measurement processes are described below.

In a basic embodiment, the pressure inside the bladder is continuously measured and the pump is activated to pass a small and fixed volume of urine (the “desired volume”) from the catheter to the drain tube every time the pressure reading is higher than a predetermined threshold. In this embodiment, every time more than some minimum volume of urine accumulates in the bladder, the pressure inside the bladder increases, triggering the pump action. The pump activation rate multiplied by the volume of each pump stroke, constitutes the UO temporal rate. Integrating temporal rate over time gives total UO values.

In preferred embodiments, urine output is measured in the following manner: Pressure inside the bladder is measured in real time with the pump not operating. During this phase, no urine flows from the bladder to the collection bag so urine accumulates inside the bladder.

When the pressure inside the bladder reaches a predetermined threshold, the pump is operated to transfer a small, fixed volume of urine from the bladder to the collection bag. After the unit volume is transferred, the pressure in the bladder is measured. If the pressure in the bladder is above a second, lower, predetermined threshold, the pump-and-remeasure process is repeated until the pressure in the bladder is below the second predetermined threshold. The process run continuously, and the quantity of urine passed at each moment is reported as the current rate, and is also integrated to give total urine output value.

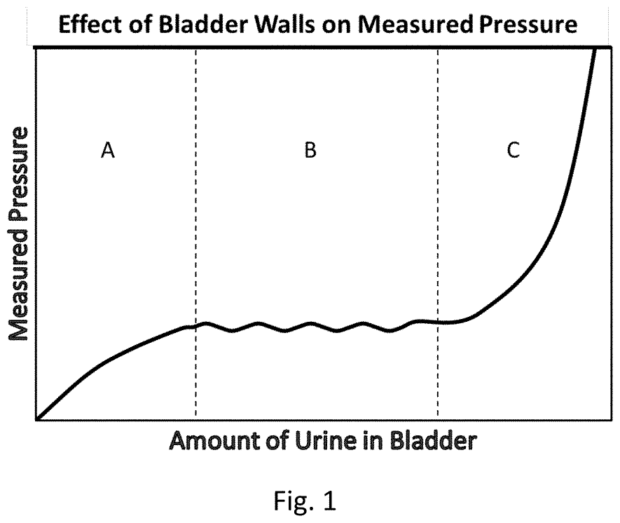

In order to accurately measure intra-abdominal pressure, the pressure in the bladder needs to be measured at a time when the bladder holds a known volume of fluid, preferably urine, although water, saline or another biocompatible liquid can be used. However, the bladder is a collapsible organ with expandable walls. As schematically illustrated in FIG. 1, when the amount of urine in the bladder is small (region “A”), the bladder walls collapse and press against each other, reducing the pressure transmitted through the bladder fluid to a pressure sensor in fluid communication with the urine; the measured pressure is lower than the IAP. In an intermediate range (region “B”), the measured pressure accurately represents the IAP. If the amount of urine is in the bladder is sufficiently large (region “C”), the bladder expands and the response of the bladder wall muscles to the expansion results in the measured pressure being higher than the IAP.

In an embodiment of the present device, an accurate measure of the IAP can be found using the following process: Starting from an empty bladder, the controller stops the pump for a period sufficient for at least a predetermined desired volume of urine to collect in the bladder. The predetermined volume is large enough to fully expand the bladder but too small to distend it. The pump is then activated to release urine through the drain tube to the collection bag; concurrently, the volume of discharged urine is measured. After the discharge of each unit volume of urine, volume of urine passed is stored and the bladder pressure is stored. After the pressure in the bladder returns to the pressure for an empty bladder, the pump is stopped. The bladder pressure at the first step in discharge process, when the bladder contained the desired volume of fluid, is reported as the measured IAP.

The process can be repeated as many times as needed, with IAP and volume of urine discharged reported each time.

A second embodiment of the method for determining IAP allows measuring both continuous Urine Output (UO) and continuous IAP, Starting from an empty bladder, the controller stops the pump for a period sufficient for at least a predetermined desired volume of urine to collect in the bladder. The predetermined volume is large enough to fully expand the bladder but too small to distend it. The pump is then activated to release urine through the drain tube to the collection bag; concurrently, the volume of discharged urine is measured and logged and the bladder pressure after the discharge of each unit volume is logged. Once pressure in the bladder shows no change for several cycles of discharge of a unit volume of urine although the bladder is not empty, the measured pressure is assigned as the current IAP. Discharge of urine is then resumed until the measured pressure is between 5% and 30% below the measured IAP value. Pumping is then stopped and pressure is monitored while more urine accumulates in the bladder. When the pressure is between 5 and 30% over the previously-measured IAP value, the cycle is repeated and a new IAP value is calculated. During this process, UO values are calculated by logging pump cycles and time.

The process can be repeated as long as necessary, with IAP and volume of urine discharge reported for each cycle.

In order to measure IAP, a different process is activated. Starting with an empty bladder as indicated by a very low pressure, the control electronics stops the pump for a pre-determined period, which is estimated by the specific patients' previously measured urine production rate to allow a little more than the desired volume of urine to accumulate in the bladder (for a normally size adult 25 cc). It then runs the pump at a controlled slow flow rate to draw urine from the bladder to the collection bag while measuring and logging the bladder fluid pressure for each measurement cycle, until the pressure reaches same very low pressure, thus building a table of pressure vs. change in bladder volume. When reaching zero pressure, the processor “looks back” at the graph of pressure/volume and determines the pressure in the bladder when it was filled with exactly the desired fluid volume, which is equal to the desired IAP measurement point. Alternatively, the software “looks back” at the graph looking for a section where the pressure was not changing despite repeated pumping actions, and reports this as the measured IAP. The process repeats indefinitely until stopped by the user.

The control electronics reports to the user the IAP measured at each cycle, as well as the actual average urine production rate and cumulative urine production rate.

In a different embodiment of the method, a slightly modified process is used in order to continually measure IAP while also measuring UO. The pump blocks the bladder allowing urine to accumulate while tracking the pressure. As the bladder fills slowly the pressure inside will increase until it is equal to the IAP, and then stay at this value for some time. When more urine accumulates, pressure will start to increase again. The processor looks for this period of time where the pressure was relatively constant, and will report this as the current IAP. Once pressure starts to rise again, the pump will release a sufficient volume of urine until the pressure goes through the plateau and starts dropping again. The volume of urine released divided by the time since last release is reported as the current UO value.

In additional embodiment of the method, the system measures UO while preserving a natural bladder voiding cycle as follows. Exit of urine from the bladder is blocked and, as urine accumulates in the bladder, the pressure rises slowly and is measured by the processor. When repetitive pressure peaks are detected, indicating naturally-occurring bladder contractions which would normally lead to urination and emptying of the bladder, the pump is activated to empty the bladder. Naturally, if pressure increases to higher than some threshold, or if time since last emptying is longer than some threshold, the pump is also activated as safety measure.

The device and method may be better explained with reference to the drawings.

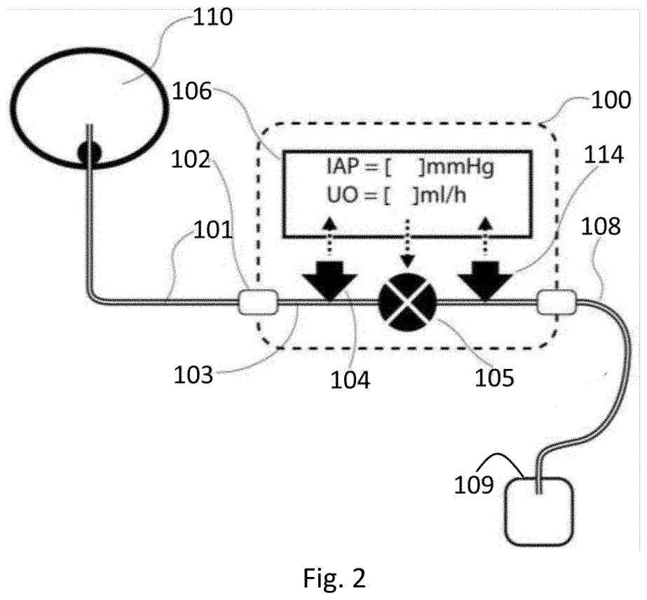

FIG. 2 describes a block diagram of a preferred embodiment of a hardware configuration needed to implement the method in a UO/IAP medical measuring device (1000).

A urine catheter 101 is inserted through the urethra into the bladder 110, and secured in place in the usual way. The lumen of the catheter is in fluid communication with the volume of the bladder, allowing urine to flow out and transmit any pressures inside the bladder to any external device in fluid communication with the lumen or through any other means. The catheter is in fluid communication with to the measuring device tube 103 by a connector 102.

The pressure inside the device tube 103 is measured by pressure transducer 104, whose port is in fluid communication with the lumen of the device tube 103. The transducer can be of any type compatible with urine, such as strain-gauge, piezo-resistive, micro-machined chip or any other element, capable of measuring pressures in the range of −50 mmHg to 100 mmHg.

The device tube 103 is connected to the inlet port of the device pump 105. The pump 105 can be any type of pump that fulfills the following requirement: A. when not activated, no urine can pass through. B. When activated, it performs the pumping of controlled and precise quantities of urine from the device tube 103, through the pump and out through the urine drain tube 108, and into the urine collection bag 109. The pump can be i.e. a peristaltic pump driven by a stepper or DC motor+encoder configuration, a piston pump with the necessary valves, or a membrane pump with two active valves.

The signal from the pressure transducer 104 is received by the device electronics 106, which can comprise a hardware circuit, a microcomputer running dedicated software, a computer processor and any combination thereof. The electronics 106 control pump operation and measurement of parameters such as, but not limited to, pressure, flow rate, volume and any combination thereof.

A second pressure transducer 114, or other mean to measure pump output port pressure, may be added to the hardware setup at the outlet port of the pump 105, with its input port in fluid communication with the lumen of the device tube 103, in order to detect blockage of the urine drain tube 108 or filling of the urine collection bag 109.

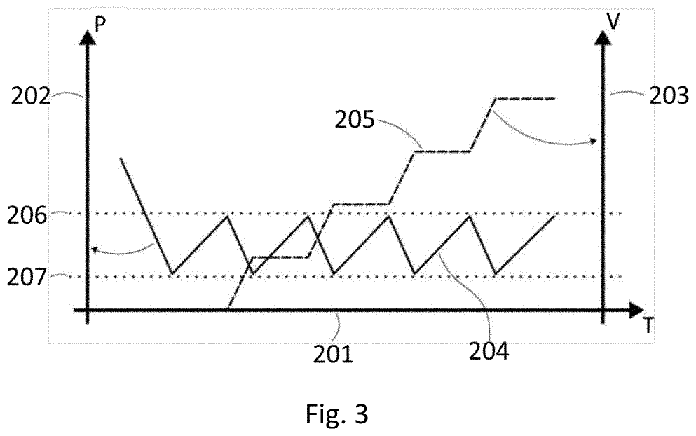

FIG. 3 schematically illustrates a typical pressure/volume vs. time graph derived from the pressure transducer, together with the measured UO value during this period in the UO continuous measuring mode when using a peristaltic pump.

The horizontal axis 201 is time, the left vertical axis 202 is pressure in the bladder, as reported by the pressure transducer 104, and the vertical axis on the right 203 is reported cumulative urine volume measured over time. The solid line 204 is pressure, referenced to the left vertical axis, and the dashed line 205 is volume, referenced to the right vertical axis.

After connecting the device tube to the catheter, if the measured pressure is higher than a first, upper threshold value, upper horizontal dashed line, 206, in a range of 3 mmHg-70 mmHg (in some embodiments, 5 mmHg - 70 mmHg), the pump is activated until the reported pressure in the bladder is lower than a second, lower threshold, lower horizontal dashed line 207, in a range of 0-15 mmHg. When the pressure is below the lower threshold 207, the pump is stopped by the control electronics and urine is allowed to accumulate in the bladder, thus slowly increasing the pressure in the bladder due to its elastic properties.

The pressure is allowed to increase until it reaches the first, higher pressure threshold 206, when the pump is activated to empty the bladder. Note that the pump has a linear volume/time throughput, so the pumping action can be stopped at any time as soon as the measured pressure reaches the second, lower threshold 207, and the pumped volume can assume any value. With each pumping session, the instantaneous and cumulative UO are recorded, as depicted by the dashed volume line 205.

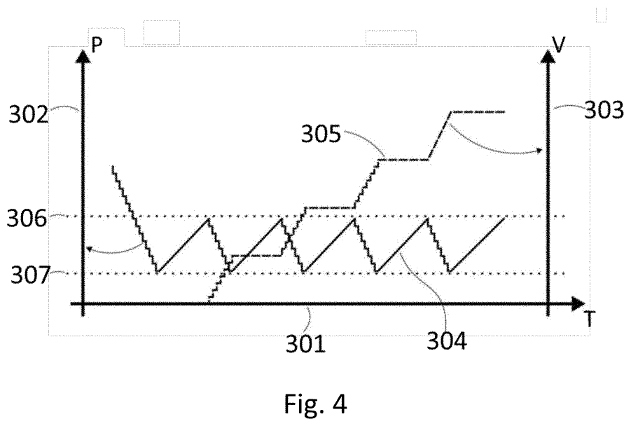

FIG. 4 Illustrates a typical pressure/volume vs. time graph derived from the pressure transducer, together with the measured UO value during this period in the UO continuous measuring mode, when using a fixed stroke volume pump.

The horizontal axis 301 is time, the left vertical axis 202 is pressure in the bladder, as reported by the pressure transducer 104, and the vertical axis on the right 203 is reported cumulative urine volume. The solid line 304 is pressure, referenced to the left vertical axis 202, and the dashed line 305 is volume, referenced to the right vertical axis 203.

After starting the system, if the measured pressure is higher than a first, upper threshold value, horizontal dashed line 306, in the range of 3 mmHg-70 mmHg (in some embodiments, 3 mmHg-70 mmHg), the pump is activated until the reported pressure in the bladder becomes lower than a second, lower threshold, lower horizontal dashed line 307, in the range of 0-10 mmHg. At this time, the pump is stopped and urine is allowed to accumulate in the bladder, thus slowly increasing the pressure in the bladder. The pressure is allowed to increase until it reaches the first, higher pressure threshold 306. Note that, since the pump transfers a fixed volume of urine in each cycle, the pumping action will only stop after the pressure has passed the lower threshold. With each pumping session, the instantaneous and cumulative UO are recorded, dashed volume line 305.

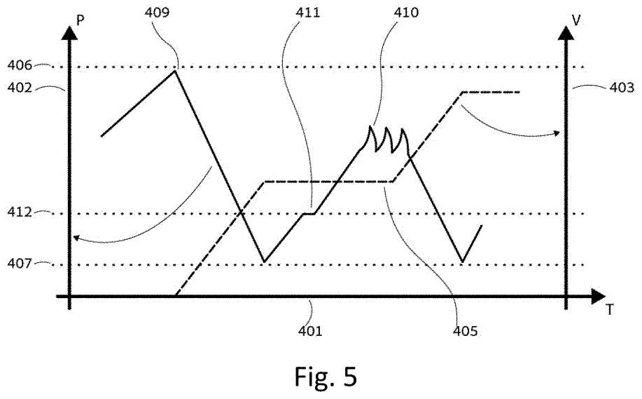

FIG. 5 illustrates a typical pressure/time graph derived from the pressure transducer, together with the measured UO value during this period, in a mode preserving a natural urination cycle. The horizontal axis 401 is time, the left vertical axis 402 is pressure in the bladder, as measured by the pressure transducer 104, and the right vertical axis 403 is measured cumulative urine volume. The solid line 411 is measured pressure, referenced to the left vertical axis 402, and the dashed line 405 is volume, referenced to the right vertical axis 403.

After starting the system, the pump is stopped until either the pressure in the bladder rises above 409 a first relatively high threshold, between 10 mmHg and 100 mmHg, the dashed horizontal line 406; or if significant cyclic fluctuations are detected in the measured pressure, indicative of repetitive contraction of the bladder in a normal voiding reflex, schematically illustrated by pressure variations 410. When either of these conditions is detected, the pump is activated until the reported pressure in the bladder is lower than a second, lower threshold in the range of 0-10 mmHg, lower dashed horizontal line 407. At this time, the pump is stopped, and the cycle repeats. The method can still report cumulative UO values, albeit less frequently, but normal urination reflexes are preserved, decreasing the risk of incontinence when catheter is finally removed.

Dashed horizontal line 412 represents the patient's IAP. It is apparent that, as urine accumulates in the bladder and the internal pressure increases, the increase in pressure will stop when the pressure inside the bladder is equal to the IAP. The pressure will remain fixed at the IAP until sufficient urine has accumulated in the bladder to totally fill it, after which the urine stretches the walls of the bladder and the IAP starts to increase. When the IAP has been reached in the bladder, the sensor will detect a period of time where the pressure remains constant 411; this pressure will be reported as the current IAP.

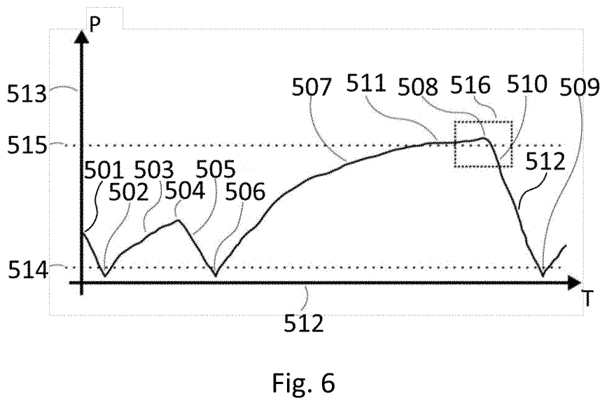

FIG. 6 schematically illustrates a typical pressure/time graph derived from a pressure transducer in an embodiment of an IAP measuring cycle. The horizontal axis 512 is time, and the left vertical axis 513 is pressure in the bladder, as measured by the pressure transducer. The method is implemented using a hardware setup as described in FIG. 2. Hardware related actions will be denoted as in FIG. 2.

Before activation, while in the system is in a stand-by state, the pump 105 is static, hence the device tube 103 is blocked and urine can't pass from the bladder to the drain tube and the collection bag. Since the air in the device tube 103 is now in fluid communication with the internal volume of the bladder, it is compressed until its pressure is equal to the pressure in the bladder at the time 501.

This increase in pressure is sensed and, if it lasts more than a few seconds, as may happen due to handling, the system will automatically switch to an active measurement mode of operation. The pump 105 is activated to drive fluids from the device tube 103 to the urine drain tube 108. As air, and then urine, are pumped from the bladder, the pressure in the bladder drops until it reaches near-zero 502.

The pump 105 is then stopped for a predetermined time, preferably in the range of 0.5-20 minutes. During this time, urine is generated by the patient and accumulates in the bladder, slowly increasing the internal pressure. This process is depicted as the rising slope 503 up to some pressure 504.

After this fixed period, the pump is reactivated and the pressure in the bladder drops again 505 as urine is pumped out until it reaches near zero again 506. Since the volume of pumped fluid for each stroke is known, it is possible to calculate the rate at which the patient is producing urine as the ratio between the volume pumped and the time-length of period 502 to 506.

Based on this urine production rate, the period is calculated in which the patient will produce a urine volume which is a larger by 10%-20% than the desired volume dictated by the accepted standard for measuring IAP for that particular patient. As urine accumulates in the bladder, the pressure in the bladder increases again 507. Note that at some point, when the pressure inside the bladder is equal to the IAP, pressure will stop increasing for a time, until the bladder is full and the walls of the bladder start stretching. In this embodiment, the pressure will continue to increase until the urine volume has reached the desired amount 508. The pump is then activated again until the pressure drops back to zero 509. As the pump is operating, a pressure measurement is stored after each fixed volume of urine is removed from the bladder, thus building a table of pressure vs. removed urine volume 512.

As soon as near-zero pressure 509 is reached, the electronics “looks back” at the graph to the point 510, where the desired volume was still inside the bladder, and marks the pressure at that point. This bladder pressure is, by the standard definition of IAP, the current IAP value, and it is presented to the user on the display on the device case 107, or transmitted by any communication means to a patient monitor or external display and alarm.

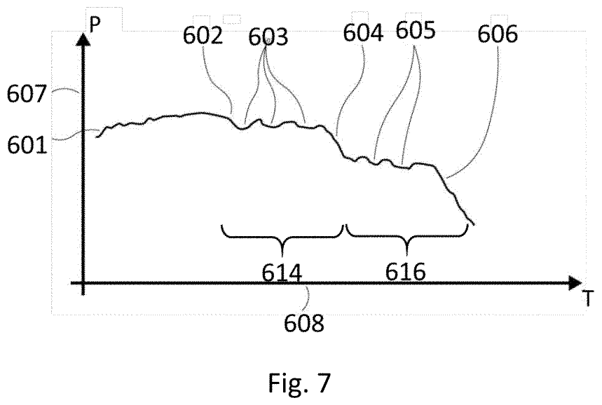

FIG. 7, schematically illustrates an enlarged view of the pressure, as shown in the dashed box 516 in FIG. 6, showing two pumping steps 614 and 616 in a typical pressure/time graph derived from the pressure transducer when using a fixed stroke pump.

It is important that the pump flow rate is low, to prevent a significant pressure drop in the tubing due to its flow resistance. A suitable pump stroke in the range of 0.1 cc-5 cc is preferable, but a constant flow pump operated intermittently and stopped between activations is also suitable.

The output from the pressure transducer can be monitored to find cyclic oscillations with a frequency of 0.01 Hz-2 Hz, which are characteristic of respiratory movements. In order to comply with the standard of practice, bladder pressure should be measured at the lowest-pressure points representing end-expiratory points of the respiration cycle. The respiratory induced pressure wave can then be analyzed and 2-4 pressure samples can be taken at the minimum pressure points 603 and 605 in two consecutive pumping steps 614 and 616, and then run the pump at the inspiratory phase in preparation for measurement at the next expiratory cycle, 604 and 606. This way, the pumping time is not a limiting factor in the overall process and the entire bladder clearing period can be completed in a few minutes.

A modification of the above method, which can speed up the process, is to proceed at a high pumping rate until the pressure drops to a value 2 mmHg-10 mmHg above the expected IAP value. At that point, the pump is switched to a low pumping rate, which can be either a step-wise pumping rate or a continuous pumping rate, to be able to accurately measure the IAP. After passing the point expected to reflect the IAP at the target volume, the pump is switched to a fast pumping rate, which can be but need not be the same as the first fast pumping rate, until the measured pressure is near zero, at which point, a slow step-wise process is resumed in order to accurately determine the zero point. For example, if previous measurements showed that over 30 minutes the patient has accumulated 30 cc of urine and the target volume is 25 cc, the next cycle will pause for 28 minutes, then proceed with a step-wise emptying process for the first 6 cc in order to get an accurate IAP measurement. The pump is then run in a continuous fashion until 20 cc has been removed, and step-wise operation is resumed for the last 2 cc in order to accurately detect the zero value. Of course, the pump flow rate or stroke volume should be stable and known in advance at all pumping speeds.

Naturally, together with the measurement of IAP, the total urine volume pumped is measured, which, when divided by the time provides a UO rate value.

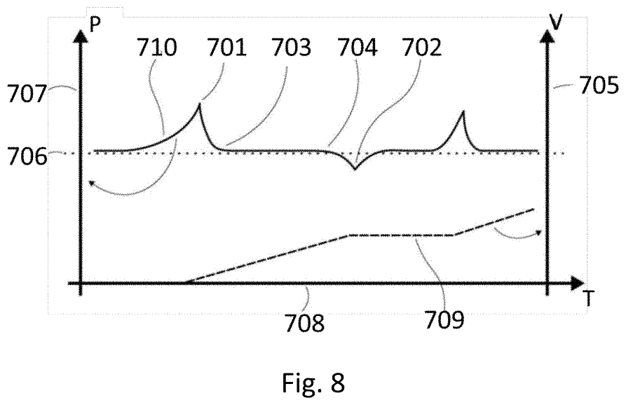

FIG. 8 illustrates an embodiment of a method for measuring both UO and IAP in a real-time, continuous mode.

FIG. 8 shows a typical graph of pressure and volume vs. time for the continuous UO+IAP measuring process of this embodiment. The horizontal axis 708 is time, the left vertical axis 707 is pressure in the bladder, as measured by the pressure transducer, and the right vertical axis 705 is cumulative volume of urine pumped out of the bladder. The solid line 710 is measured pressure, left vertical axis 707, and the dashed line 709 is measured volume, right vertical axis 705. The method is implemented using a hardware setup as described in FIG. 2. Hardware related actions will be denoted as in FIG. 2.

By blocking the flow of urine out of the bladder and allowing urine to accumulate, the urine volume inside the bladder slowly increases. As time passes, the urine volume inside the bladder slowly increases until the bladder reaches its full, non-stretched volume. At this point, the pressure measured by the pressure sensor fluidly coupled with the internal volume of the bladder through the catheter starts to increase. When the pressure has increased to a value 5%-30% higher than the previously measured IAP 701, the pump is activated, drawing urine out of the bladder at a rate faster than the rate of urine production. As the volume of urine inside the bladder decreases, pressure returns to IAP 703. The pressure remains about the same until the bladder begins to collapse and the pressure begins to drop 704. The pump continues to operate until the pressure reaches a value 5% to 30% less than the last measured IAP, 702 (typically 0-5 mmHg), when the pump is stopped and the cycle repeats. The horizontal dotted line 706 represents the measured IAP, and the dotted ramp 709 represents the cumulative urine volume referenced to the right vertical axis 705. Assuming a normal urine production rate of 2000 cc/day and an optimal IAP measuring bladder volume of 20-30 cc, the cycle will repeat approximately every 10-20 minutes.

What is claimed is:

1-30. (canceled)

31. A device for measuring a rate of production of urine in a patient comprising:
a pump;
a catheter connected to said pump and configured for connecting in fluid communication to a bladder of said patient;
a pressure sensor configured to measure a pressure in said catheter; and
a processor in communication with said pressure sensor and configured to operate said pump to perform at least one urine output measurement cycle, said cycle comprising:
(i) occluding said catheter for a first time period until a predetermined first pressure within said catheter is detected,
(ii) emptying said bladder over a second time period until a predetermined second pressure within said catheter is detected,
(iii) measuring a volume of urine discharged from said bladder as a result of said emptying, and
(iv) calculating said rate of production of urine in said patient as equal to said volume divided by a sum of said first and second time periods.

32. The device of claim 31, wherein said operating comprises performing at least two of said cycles consecutively.

33. The device of claim 32, wherein, with respect to any consecutive pair of said cycles, said rate of production is equal to said measured volume associated with a first cycle of said pair of said cycles, divided by a sum of:
said second time period associated with said first cycle of said pair of said cycles; and
(ii) said first time period associated with a second cycle of said pair of said cycles.

34. The device of claim 31, wherein said first time period has a predetermined duration.

35. The device of claim 31, wherein said predetermined first pressure is in a range from 3 mmHg to 70 mmHg and/or said predetermined second pressure is in a range from 0 mmHg to 5 mmHg.

36. The device of claim 31, wherein said processor is further configured to operate said pump to perform intra-abdominal pressure (IAP) measurement cycle, said IAP measurement cycle comprising:
(i) occluding said catheter for a third time period until said bladder holds a volume of urine above a desired threshold volume, wherein said third time period is determined based, at last in part, on said calculated rate of production of urine;
(ii) emptying said bladder in a plurality of steps, wherein each of said steps discharges a specified volume of urine;
(iii) measuring said pressure in said catheter associated with each of said steps; and
(iv) determining said IAP as said measured pressure associated with a step of said steps wherein said volume of urine is equal to said desired threshold volume.

37. The device of claim 36, wherein each of said steps lasts at least two breathing cycles of said patient, wherein said measuring is performed continuously during each respective step, and wherein said measured pressure is equal to the lowest value of said measuring during each respective step.

38. The device of claim 36, wherein said third time period is predetermined.

39. The device of claim 36, wherein said desired threshold volume is determined as a desired threshold volume for optimal IAP measuring.

40. The device of claim 39, wherein said desired threshold volume for optimal IAP measuring is a desired threshold volume for optimal IAP measuring in said patient.

41. A method for measuring a rate of production of urine in a patient comprising:
providing a device comprising:
a pump,
a catheter,
a pressure sensor configured to measure a pressure in said catheter, and
a processor in communication with said pressure sensor and configured to operate said pump;
connecting said catheter to said pump and in fluid communication to a bladder of said patient; and
performing at least one urine output measurement cycle, said cycle comprising using said processor to operate said pump to:
(i) occlude said catheter for a first time period until a predetermined first pressure within said catheter is detected,
(ii) empty said bladder over a second time period until a predetermined second pressure within said catheter is detected,
(iii) measure a volume of urine discharged from said bladder as a result of said emptying, and
(iv) calculate said rate of production of urine in said patient as equal to said volume divided by a sum of said first and second time periods

42. The method of claim 41, wherein said performing comprises performing at least two of said cycles consecutively.

43. The method of claim 42, wherein, with respect to any consecutive pair of said cycles, said rate of production is equal to said measured volume associated with a first cycle of said pair of said cycles, divided by a sum of:
(i) said second time period associated with said first cycle of said pair of said cycles; and
(ii) said first time period associated with a second cycle of said pair of said cycles.

44. The method of claim 41, wherein said first time period has a predetermined duration.

45. The method of claim 41, wherein said predetermined first pressure is in a range from 3 mmHg to 70 mmHg and/or said predetermined second pressure is in a range from 0 mmHg to 5 mmHg.

46. The method of claim 41, wherein said performing further comprises performing an intra-abdominal pressure (IAP) measurement cycle comprising operating said pump to:
(i) occlude said catheter for a third time period until said bladder holds a volume of urine above a desired threshold volume, wherein said third time period is determined based, at last in part, on said calculated rate of production of urine;
(ii) empty said bladder in a plurality of steps, wherein each of said steps discharges a specified volume of urine;
(iii) measure said pressure in said catheter associated with each of said steps; and
(iv) determine said TAP as said measured pressure associated with a step of said steps wherein said volume of urine is equal to said desired threshold volume.

47. The method of claim 46, wherein each of said steps lasts at least two breathing cycles of said patient, wherein said measuring is performed continuously during each respective step, and wherein said measured pressure is equal to the lowest value of said measuring during each respective step.

48. The method of claim 46, wherein said third time period is predetermined.

49. The method of claim 46, wherein said desired threshold volume is determined as a desired threshold volume for optimal TAP measuring.

50. The method of claim 49, wherein said desired threshold volume for optimal IAP measuring is a desired threshold volume for optimal IAP measuring in said patient.
